(19) United States
(12) Patent Application Publication
(10) Pub. No.: US 2010/0160781 A1
(43) Pub. Date: Jun. 24, 2010

(54) DOPPLER AND IMAGE GUIDED DEVICE FOR NEGATIVE FEEDBACK PHASED ARRAY HIFU TREATMENT OF VASCULARIZED LESIONS

(75) Inventors: Stephen Carter, La Conner, WA (US); Lawrence Crum, Bellevue, WA (US); Peter Kaczkowski, Seattle, WA (US); John Kucewicz, Seattle, WA (US); Mike Bailey, Seattle, WA (US); Steve Langer, Saint Charles, MN (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 12/634,599

(22) Filed: Dec. 9, 2009

Related U.S. Application Data
(60) Provisional application No. 61/120,974, filed on Dec. 9, 2008.

(51) Int. Cl. A61N 7/00; A61B 8/00

(52) U.S. Cl. 600/439

(57) ABSTRACT

A noninvasive technique that can be used to deny blood flow to a particular region of tissue, without the inherent risks associated with invasive procedures such as surgery and minimally-invasive procedures such as embolization. Blood flow in selected portions of the vasculature can be occluded by selectively treating specific portions of the vasculature with high intensity focused ultrasound (HIFU), where the HIFU is targeted Doppler ultrasound data, and a duration of the therapy is automatically controlled using a negative feedback loop provided by Doppler ultrasound data collected during the HIFU therapy. A portion of the vasculature providing blood flow to the undesired tissue is selected by a clinician, or automatically selected based on Doppler data, and HIFU is administered to the selected portion of the vasculature to occlude blood flow through that portion of the vasculature.

### RELATED APPLICATIONS

This application is based on a prior copending provisional application, Ser. No. 61/120,974, filed on Dec. 9, 2008, the benefit of the filing date of which is hereby claimed under 35 U.S.C. § 119(e).

### GOVERNMENT RIGHTS

This invention was made with U.S. government support under contract number SMS00402 awarded by the National Space Biomedical Research Institute. The U.S. government has certain rights in the invention.

### BACKGROUND

High Intensity Focused Ultrasound (HIFU) holds great potential for medical therapy. HIFU uses focused, high intensity ultrasound to selectively heat and destroy tissue. HIFU is being researched or used clinically for a range of applications including necrosis of uterine fibroids, prostate tissue, and cancer of the prostate, liver, kidney, breast, and pancreas, and opening of the blood-brain barrier.

Therapeutic uses of HIFU have generally been directed at destroying undesired masses of tissue by directly targeting the tissue itself. However, the focal region of a HIFU transducer is relatively small (approximately the size of a grain of rice). Thus, to treat the entire volume of even a relatively small tumor with HIFU to necrose the tumorous tissue requires constantly changing the position of the focal region of the HIFU transducer relative to the tumor, leading to relatively long treatment times, and requiring relatively complicated targeting systems. It would be desirable to provide a technique for utilizing HIFU's ability to non-invasively destroy undesired tissue, such as a tumor, without requiring treatment of the entire volume of the undesired tissue.

### SUMMARY

This application specifically incorporates by reference the disclosures and drawings of each patent application and issued patent identified above as a related application.

The present disclosure relates to the destruction of undesired tissue by selectively targeting vasculature providing nutrients to the undesired tissue. According to the techniques described herein, blood flow in selected portions of the vasculature can be occluded by selectively treating specific portions of the vascular system with HIFU. By denying undesired tissue the nutrients and oxygen provided by blood flow, the techniques described below will cause necrosis in the undesired tissue, thereby reducing the volume of such undesired tissue, or eliminating the undesired tissue.

Significantly, Doppler ultrasound imaging is employed to analyze the blood flow into the undesired tissue. Based on the Doppler flow data, a portion of the vascular identified as providing the greatest flow of nutrients into the undesired tissue is selected as the target location. The focal point of the HIFU transducer is directed toward the target location, and HIFU therapy is initiated while continuing to image the target location. Simultaneous or real-time ultrasound imaging of HIFU therapy can be achieved using one or more of the techniques disclosed in U.S. Pat. No. 6,425,867 (disclosing a gating technique); U.S. Pat. No. 7,621, 873 (disclosing another gating technique); and U.S. Patent Application Publication No. 2006-0264748 (disclosing a software based technique). Techniques for verifying the HIFU focal point in an ultrasound before initiating HIFU therapy are disclosed in U.S. Pat. No. 6,425,867 (disclosing energizing the HIFU beam at a relatively low power to change the echogenicity of tissue at the focal point without damaging the tissue) and U.S. Patent Application Publication No. 2005-0038340 (disclosing introducing an ultrasound contrast agent to the target location, and energizing the HIFU beam at a relatively low power to visualize the ultrasound contrast agent at the focal point, without damaging the tissue proximate the focal point).

In one exemplary embodiment, a combined display is provided to the user, wherein the Doppler ultrasound image data and the focal point of the HIFU beam are simultaneously displayed. The display can include dosage information relating to the relative amount of HIFU energy delivered to the target location. The display can include flow rate information for the vascular structures displayed in the Doppler ultrasound image. The display can include targeting crosshairs for the HIFU focus, and an icon indicating the status of the HIFU beam (i.e., on or off).

In an exemplary embodiment, once a clinician has identified the specific vascular structure to be targeted, the HIFU beam is automatically steered to the target location and HIFU therapy is initiated, and continued until the Doppler data from that vascular structure indicates that the blood flow rate has dropped to a predetermined value. The predetermined value can vary, and in at least one embodiment, the clinician can define the predetermined value. Exemplary, but not limiting predetermined values include a flow rate reduction of 100%, a flow rate reduction of 90%, a flow rate reduction of 75%, a flow rate reduction of 50%, and a flow rate reduction of 25%. In general, a larger flow rate reduction is more desirable (as this will deny the undesired tissue a greater quantity of nutrients), although the clinician may determine that less of a reduction is desired in a particular treatment. If desired, the clinician can manually position the focal point of the HIFU beam, such that automatic control of the HIFU therapy is not enabled until the clinician is satisfied that the HIFU focal point is properly positioned. Preferably, a cut off switch is provided to enable the clinician to terminate the automated HIFU therapy at anytime.

The automated HIFU therapy can be considered to utilize a negative feedback loop, in that as the therapy progresses and blood flow at the target location is reduced, that reduction is reflected in the Doppler ultrasound data. Once the reduction matches the predetermined value, the HIFU therapy is automatically halted. If the clinician has identified more than one different vascular structure, the automated HIFU therapy will continue until each selected vascular structure has been treated.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWING

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a flowchart illustrating the logical steps implemented in a method for using HIFU therapy to the vascular system in order to treat undesired tissue;

FIG. 2 is a functional block diagram of an exemplary system for implementing the concepts disclosed herein;

FIG. 3 schematically illustrates an exemplary image combining ultrasound image data of a vascular structure providing nutrients to undesired tissue, Doppler data indicating blood flow, a HIFU targeting icon, HIFU dosage data, and a HIFU status icon;

FIG. 4 schematically illustrates an experimental setup employed in an empirical study to test the concepts disclosed herein;

FIG. 5 schematically illustrates the system employed in the empirical study; and

FIG. 6 graphically presents data collected in the empirical study.

### DESCRIPTION

### Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

Disclosed herein is a system using transcutaneous HIFU to heat or denature bio-molecules for cauterization, vascular ablation, or tissue necrosis, using ultrasound image guidance. The system can provide a less invasive technique, as compared to surgery, embolization, and endoscopy, for both treatment and de-bulking of benign or malignant vascularized masses.

Imaging modalities used for targeting and monitoring are Doppler based (power Doppler or directional color flow). The HIFU and imaging systems are mechanically and acoustically registered prior to the treatment, and the imaging display includes location of the HIPU focal zone. Targeting control is shared between the clinician and the system, and can become more automated as target tracking algorithms are refined. A therapeutic region of interest (ROI) is defined by the clinician using ultrasound displaying control system, within which the energetic Doppler signals are used to aim the HIFU beam by electronic array steering (and/or mechanical steering). Doppler ultrasound imaging will also be used to monitor the progress of the treatment and provide feedback to the HIFU delivery system for dose control.

The anticipated benefits and aims are: (a) a significant decrease of HIFU dose over whole lesion treatment volume by targeting supplying vessels only; (b) a corresponding reduction in treatment time; (c) implementation of an outpatient procedure with decreased morbidity and expense to the patient; (d) a simplified procedure for the physician with a combination of semiautomatic therapy control and target acquisition (via reduced signal from Doppler target; i.e.: fading color signature).

In a first exemplary embodiment, the application of HIFU therapy will be largely directed by the human operator. Exemplary steps include: (A) scanning of the patient to locate the Doppler signal in the supplying vessels; (B) selection of the region of interest around the target vessels; (C) execution of a HIFU target overlay onto the anatomical image; (D) operator initial selection of HIFU power level; (E) operator initiated HIFU pulse (indicated by “therapy on” light); (F) assessment of outcome from diagnostic image and Doppler signal; and (G) repeat as required.

Visually, the operator would see the blood flow as a streaming red or blue target in the region of interest. Through the use of a control such as a mouse or joystick (or other interface), the operator would place a cross hair on the target, and adjust the size of volume to receive therapy (possibly with a thumb wheel that increases the radius of treatment). After selecting the target center and radius, a therapy switch would be actuated to deliver the HIFU pulse.

In a partially automated second exemplary embodiment, steps A-E above would be performed as written, but F and G would be performed automatically by HIFU therapy algorithms. Such algorithms would terminate therapy when the background Doppler signal, and that in the region of interest, come into arbitrarily close agreement. However, at all times the operator would have the option to override the system.

In a still more automated third exemplary embodiment, steps A-B above would be performed as written, but C-G would be performed automatically by HIFU therapy algorithms. Again, such algorithms would terminate therapy when the background Doppler signal, and that in the region of interest, come into arbitrarily close agreement. However, at all times the operator would have the option to override the system. In an even more automated embodiment, the clinician would identify the abnormal treatment to be treated, and the system will automatically identify the vascular structure providing the majority of the blood flow into the abnormal tissue, and target that structure. The negative feedback loop from the Doppler data would control the duration of the HIFU therapy.

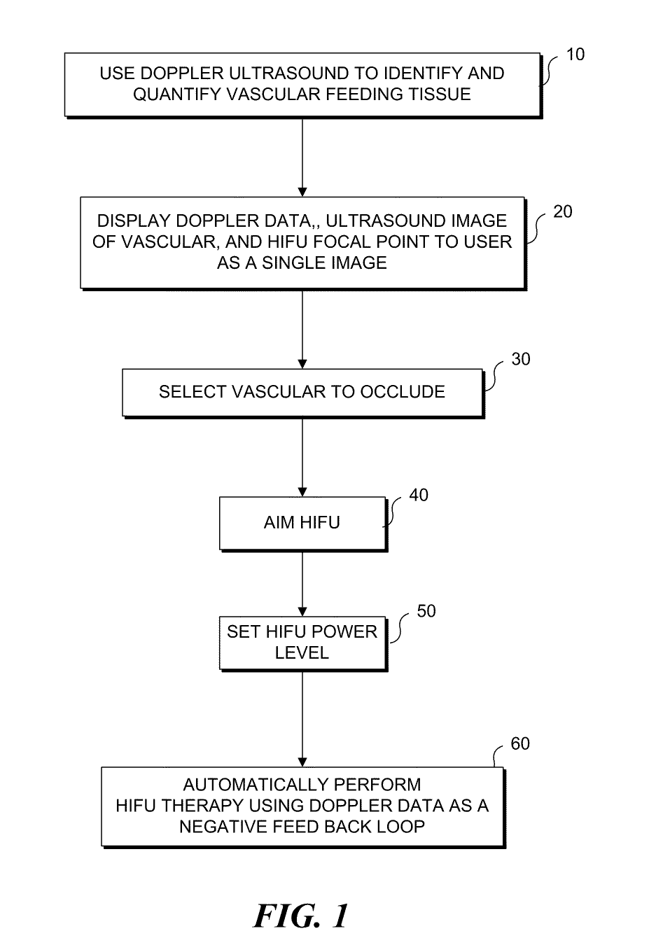

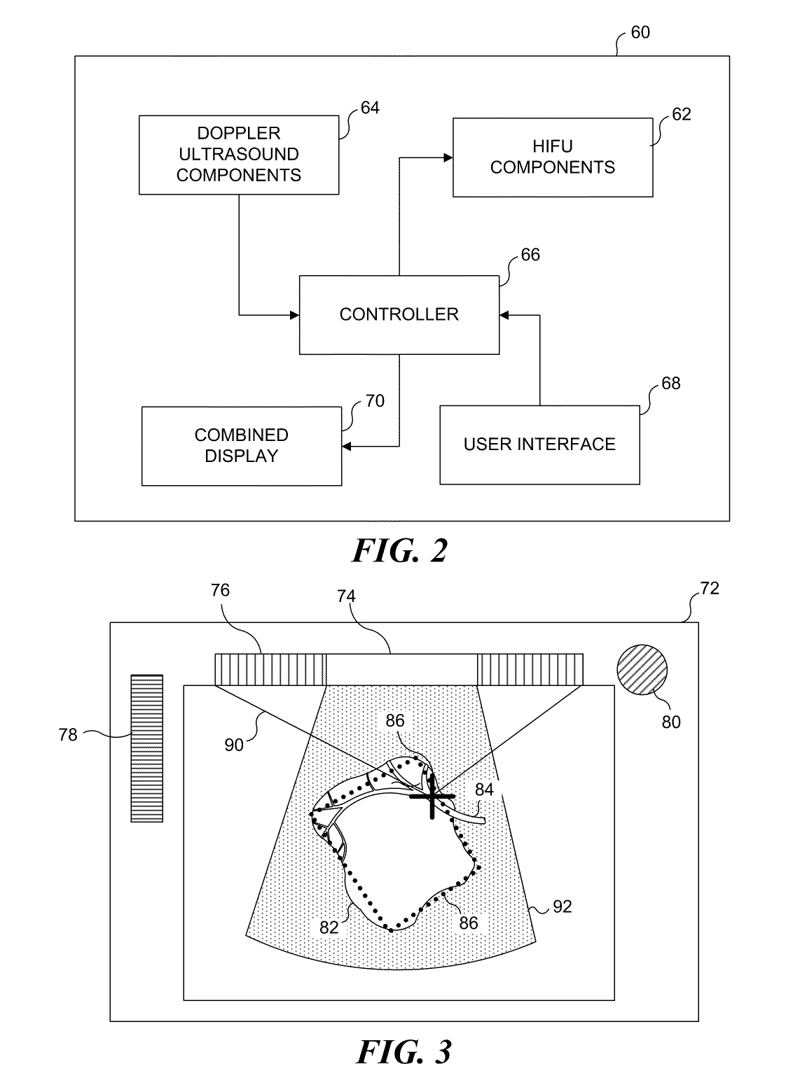

FIG. 1 is a sequence of logical steps to perform HIFU therapy on the vascular system to treat undesired tissue. As noted above, such therapy can be used to de-bulk or eliminate tumors by denying nutrients provided by blood flow. In a block 10 an image of the vascular structures providing blood flow into the unwanted tissue is obtained using Doppler ultrasound imaging. In a block 20 the Doppler data is displayed to a user in a combined image (see FIG. 2) that includes the Doppler data providing information about blood flow (generally displayed using color), an ultrasound image of the vascular structure feeding the undesired tissue (such as a B-mode image), and the focal point of the HIFU transducer. The display preferably also includes a HIFU status indicator and a HIFU dosage indicator. This combined image is updated frequently (as often as practical, preferably with a refresh rate of 24-30 frames per second or more; understanding that lower refresh rates may be useful, but will provide a lower quality display), and provides real time imaging of the treatment site.

In a block 30 a particular portion of the vascular system associated with the unwanted tissue is selected as a treatment site. Preferably a clinician will exercise care in selecting an appropriate treatment site, to ensure that the treatment site selected does not provide blood flow to vital organs or healthy tissue that is to remain unaffected by the treatment. The vascular structures selected as a treatment site can be fully or partially encompassed by the undesired tissue, or can be spaced apart from the undesired tissue. Where the vascular structure is not encompassed by the treatment site, the clinician will need to pay particular attention to ensuring that occlusion of the vascular structure will not detrimentally affect vital organs or healthy tissue, which should not be damaged by the therapy. Those of ordinary skill in the art will recognize that the particular vascular structures selected as a treatment site will be a function of the type and location of the undesired tissue being treated. An exemplary implementation of this technique will be its use as an alternative to uterine artery embolization (an invasive therapy used to de-bulk uterine fibroids by occluding blood vessels providing nutrients to the fibroid). In such an implementation, the treatment site will be branches of the uterine artery primarily servicing the fibroid itself. Thus, the treatment sites will generally be located relatively close to the uterine fibroid, or within the uterine fibroid, to prevent interruption of blood flow to other portions of the uterus. Particularly because of the potential negative implications of occluding blood flow to healthy tissue or vital organs, those of ordinary skill in the art will readily recognize that the step of choosing an appropriate treatment site must be carried out very carefully. The treatment site will therefore normally be selected to maximize a beneficial therapeutic effect, while minimizing any undesired effects. Thus, selection of a treatment site will generally be based not only on a thorough knowledge of anatomy and the vascular system, but also on a careful review of the particular patient's vascular system in the affected area, to help ensure that the selected treatment site does not provide blood flow to a vital organ or other tissue that should not be damaged.

In one embodiment, the clinician manipulates a user interface (such as a mouse, joystick, or touch screen, understanding that other types of interfaces, can also be employed) to specifically define the location where of the HIFU focal point. In such an embodiment, the clinician is examining the displayed Doppler data and determining the appropriate treatment site.

In a related embodiment, the clinical defines a region of interest (ROI) that may include more than one vascular structure. In such an embodiment, the clinician is examining the displayed Doppler data and determining a relatively larger volume in which administration of HIFU will not negatively effect healthy tissue. The system then can process the Doppler data to determine what locations within the ROI correspond to vascular structures providing blood flow into the undesired tissue (the system processes the Doppler data to identify vascular structures in the ROI having the highest flow rates; i.e., the greatest Doppler signal), and treat those structures automatically using a negative feed back loop based on the Doppler signal to determine when the therapy is complete. The clinician can view the automated treatment in real-time on the display, and the clinician can terminate the automated therapy at any time. Where the ROI includes multiple vascular structures, many different control algorithms can be employed. In one exemplary embodiment, the system uses the Doppler data to rank the vascular structures in order of their relative flow rates, and treats the vascular structure contributing the most flow first. Recognizing that some undesired vascularized tissue masses may have many relatively small vascular structures providing nutrients, as well as several relatively larger vascular structures providing nutrients, the control algorithm can be configured to ignore relatively small vascular structures, and treat only the relatively larger vascular structures (for example, vascular structures providing less than 10% of the blood flow into the ROI can be ignored, recognizing that the 10% figure is exemplary, and not limiting).

Referring once again to FIG. 1, in a block 40 the HIFU transducer is aimed at the target; that is, the focal point of the beam is selectively positioned to correspond to the target. As noted above, the aiming process can be performed manually by the clinician, or automatically by the system (based on the Doppler signal, as discussed above). If desired, the position of the focal point can be verified by energizing the HIFU beam at a relatively low power level and examining the display to identify changes in the ultrasound image indicating the position of the focal point. In a block 50, the clinician sets the power level of the HIFU. In some embodiments, this step can be skipped, and a default setting is employed.

In a block 60, the system automatically controls the HIFU component to perform the therapy, targeting sites specified by the clinician or sites automatically selected in a clinician defined ROI (the automated selection is based on the Doppler signals from the ROI). Again, the clinician views the therapy in real-time on the display, and can terminate the automated therapy at any time.

FIG. 2 is a functional block diagram of an exemplary system 60, which includes a HIFU component 62, a Doppler ultrasound component 64, a controller 66, a user interface 68, and a display 70. HIFU component 62 is intended to represent the HIFU transducer, as well as the additional equipment required to energize the transducer. Doppler ultrasound component 64 is intended to represent the imaging ultrasound transducer, as well as the additional equipment required to energize the imaging transducer. Not specifically shown are the elements required to enable real-time ultrasound imaging during HIFU therapy to be implemented (requiring gating control elements or software elements). As noted in the Summary, those elements are described in patents and published patent applications specifically incorporated herein by reference. Controller 66 can be implemented using a computing device (software based) or a custom circuit (hardware based). Controller 66 drives display 70, responds to user input, controls the Doppler component and the HIFU component, and implements any automatic functions disclosed herein (such as selecting target locations in a user defined ROI based on Doppler flow data, and terminating therapy based on negative feedback provided by the Doppler data). It should be recognized that controller 66 can be implemented by more than one component working in concert (i.e., a separate processor/controller can be associated with one or more of HIFU component 62, a Doppler ultrasound component 64, and a display 70).

In an exemplary but not limiting embodiment, HIFU component 62 is an annular array transducer, with a central orifice into which Doppler ultrasound component 64 can be inserted, to facilitate registration and alignment of the imaging plane and HIFU therapy beam.

FIG. 3 schematically illustrates an exemplary combined display 72, which provides a user an ultrasound image 92, Doppler data (not specifically shown, noting that Doppler data generally is presented to a user as colorized portions of an ultrasound image, where different colors represent different flow intensities), and the relative position of a HIFU focal point (indicated by a crosshair 86) on a single display. The combined display is updated during therapy, to enable the clinician to monitor (and override if required) the automated portions of the therapy.

Optional elements in combined display include a HIFU power icon 80 that enables the clinician to quickly determine if the HIFU beam is or is not energized, and a HIFU dosage icon 78 that changes over time during the therapy to provide a relative indication of how much HIFU energy has been delivered to a particular target location. Optional icons 74 and 76 indicate the locations of the Doppler ultrasound transducer and the HIFU transducer. A graphical element 90 indicates the beam shape of the HIFU transducer relative to the ultrasound image.

As shown, ultrasound image 92 in display 72 shows a mass of tissue 82, including a vascular structure 84. A clinician has used a user interface to define a ROI 86 that substantially encompasses the tissue mass. As described above, the clinician can use the user interface to place crosshairs 86 at a particular portion of vascular structure 84 to be treated, and allow the automated therapy to proceed at the user defined location. Alternately, having defined the ROI, the clinician can allow the automated system to use the Doppler data to determine the region of greatest flow in vascular structure 84, and automatically target that location during therapy. If while monitoring the automated therapy, combined display 72 indicated to the user that a HIFU dosage has exceeded a predetermined value, or that the HIFU focal point no longer corresponds to a user defined target or is no longer in the user defined ROI, the clinician can terminate the automated therapy.

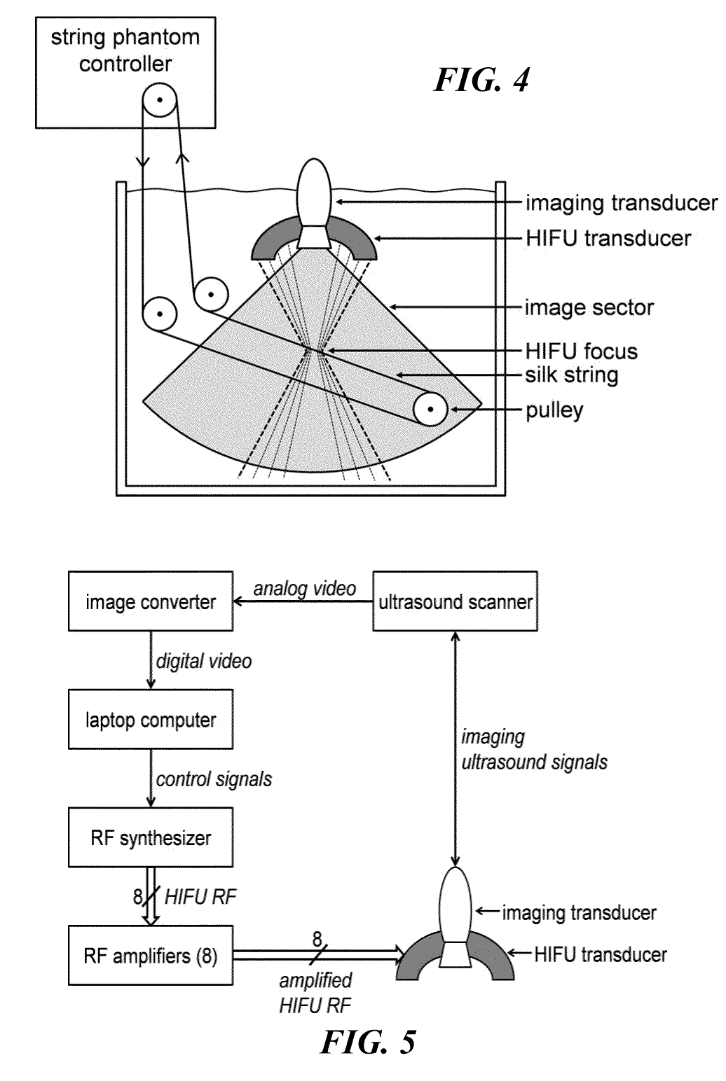

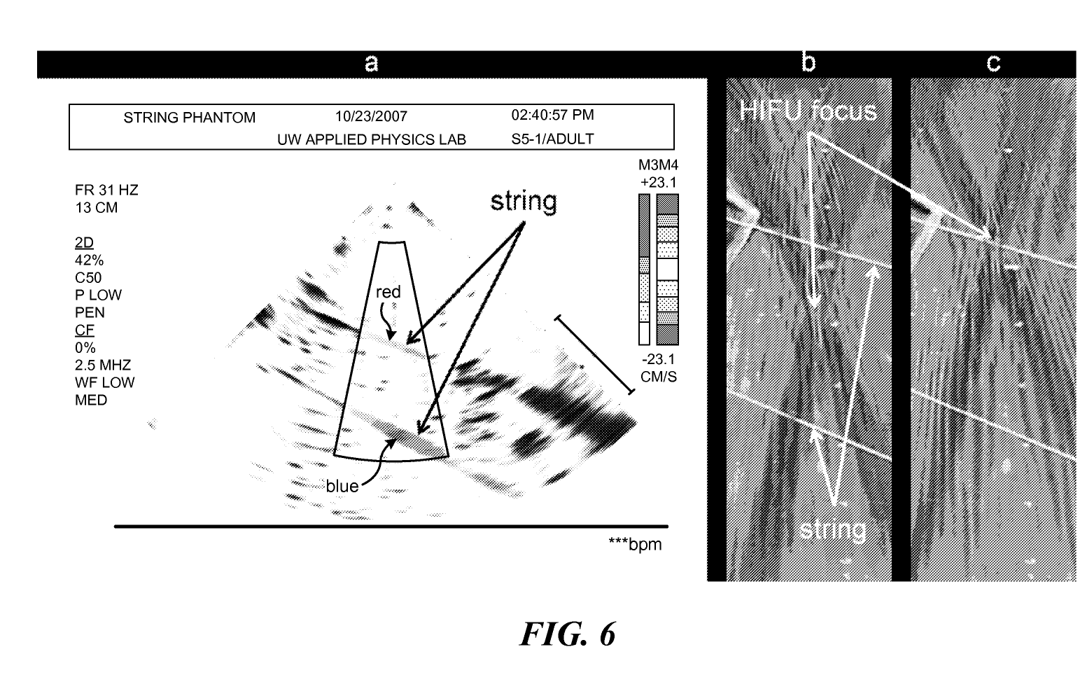

FIGS. 4, 5 and 6 relate to an empirical study implemented to prove that Doppler signals can be used to automatically target a HIFU beam. FIG. 4 schematically illustrates an experimental setup employed in the empirical study, FIG. 5 schematically illustrates the system employed in the empirical study, and FIG. 6 graphically presents data collected in the empirical study.

In that empirical study, Doppler ultrasound was employed to target HIFU onto a moving phantom (a vibrating string) simulating a blood vessel. HIFU was delivered using a PZT (lead zirconium titanate) annular array transducer (Sonic Concepts, Bothell, Wash., USA) with an aperture of 6.3 cm and a radius of curvature of 6.2 cm, transmitting at 2.75 MHz. The transducer included eight (8) concentric elements of equal area around a central opening with a diameter of 2 cm, the opening accommodating a phased array imaging transducer. The HIFU transducer was driven using a SC-200 radiofrequency (RF) synthesizer (Sonic Concepts) that allowed for the independent control of the amplitude, phase, and frequency of the ultrasound signal transmitted by each transducer element. By appropriately setting the phase of the signal delivered to each element, the depth of focus could be adjusted axially from 4.5 cm to 7.5 cm. Eight independent amplifiers (IC-706MKIIG, Icom America Inc., Bellevue, Wash., USA) were used to amplify the signal delivered to each transducer element.

The HIFU system produced a focal intensity greater than 5000 W/cm², which is within the common therapeutic range, and which has been tested by US Army surgeons in animal acoustic hemostasis experiments simulating battlefield trauma. An iE-33 ultrasound scanner (Philips Medical Systems, Bothell, Wash., USA) with a 4V2 phased array imaging transducer was used to collect Color Doppler ultrasound data. A custom fixture was built to hold the imaging transducer and couple it with the HIFU transducer, allowing imaging through the central opening in the HIFU transducer. When coupled, the central scan line from the two-dimensional sector imaged by the ultrasound scanner corresponds with the HIFU transducer's axis of ultrasound propagation.

A laptop computer (Dell Inspiron B130, Dell Inc., Round Rock, Tex., USA) running LabVIEW (National Instrument Corp., Austin, Tex., USA) was used to acquire data from the ultrasound scanner, process the data to detect a motion (i.e., the simulated blood flow), and control the HIFU system. When instructed by the operator, LabVIEW captures the image currently displayed on the ultrasound scanner from the video output on the back of the ultrasound scanner, using an image converter (DFG/USB2-1t, The Imaging Source LLC, Charlotte, N.C., USA).

Doppler signals from vessels with blood flow (bleeding or intact) have unique signatures that will be incorporated into a bleeding detection algorithm. For proof-of-principle testing using the string phantom to simulate blood flow, a simple algorithm was implemented to detect either the greatest positive or negative velocity, as selected by the operator, in the region of the ultrasound image corresponding to the focal range of the HIFU transducer. With Color Doppler enabled, the ultrasound scanner displays a gray-scale B-mode image showing tissue structure along with the superimposed Color Doppler image showing velocity. Color Doppler information is only displayed where the local velocity exceeds a threshold velocity determined by a combination of controls on the ultrasound scanner. After capturing the image displayed on the ultrasound scanner, the detection algorithm extracts the line of pixels in the 640×480 RGB-encoded (red, green, blue) image corresponding to the focal range of the HIFU transducer along with the pixels in the Color Doppler color scale. B-mode pixels, i.e. pixels with equal red, green, and blue values, were discarded and each of the remaining pixels in the HIFU focal range was mapped to the most similar pixel in the Color Doppler color scale to determine its velocity. The location of the pixel with either the greatest positive or negative velocity is identified to be the location of the string phantom. After the string phantom was detected, the LabVIEW program instructed the SC-200 RF synthesizer to change the focus of the HIFU transducer and display the location of the focus on the captured ultrasound scanner's image. The combined time to detect the string phantom and reprogram the HIFU focus is on the order of one second. From the operator's view, the coupled transducers are placed over the target, a Doppler image is acquired, the system detects the target and displays its location superimposed on the Doppler image, and the system focuses the HIFU transducer on the target. If the operator concurs with the target location, HIFU can be enabled using a foot pedal switch.

The empirical system was tested using a Mark III Doppler string phantom (JJ&A Instruments, Duvall, Wash., USA), a standard device for simulating blood flow for calibrating and testing Doppler imaging. A system of pulleys was used to guide a silk string (Genzyme Biosurgery, Fall River, Mass., USA) through a transparent acrylic tank with degassed water. The coupled HIFU and imaging transducers were submerged in the water and positioned using B-mode imaging to visualize the string moving in two directions. The coupled transducers were held in place using an articulated clamp (Manfrotto, Bassano del Grappa, Italy). In this empirical test, the ultrasound scanner was configured manually. After positioning the transducers, Color Doppler was activated and the operator adjusted the sampling frequency, the Color Doppler and B-mode gain, and the write priority to image the moving string without aliasing.

A Schlieren imaging system built in-house was used to visualize the HIFU acoustic field to verify the targeting and delivery of HIFU. Schlieren imaging does not have a role in the therapy or device described herein, other than to provide a clear visualization of the targeting accuracy for the empirical study. Schlieren imaging is an optical technique permitting visualization of sound waves in a transparent medium. Collimated light is directed through the water tank and refracted by the change in water density due to the HIFU sound field creating an image of the sound field.

The system successfully detected the string and adjusted the HIFU focus to target the string.

FIG. 6 graphically presents data collected in the empirical study. Portion A shows an image from the ultrasound scanner showing the string in blue moving away from the ultrasound transducer, and after passing through a pulley returning in the opposite direction in red. The image is very similar to what would be seen if imaging an artery and a vein in the body. Portion B of FIG. 6 shows a Schlieren image with the string and the hourglass-shaped HIFU field prior to detection and targeting. In this image, the waist of the hourglass, the HIFU focus, occurs between the two string segments. After being instructed by the operator to target the highest positive velocity, the system successfully detected and refocused the HIFU transducer on the upper string segment as indicated in Portion C of FIG. 6 by the intersection of the string and the waist of the hourglass. The string segment moving in the opposite direction was also detected and successfully targeted. Without the string phantom running, the system also correctly determined and alerted the operator that there was no detectable target present.

In the empirical system, Color Doppler data is collected using a general-purpose ultrasound scanner that was not designed to be remotely controlled. In the empirical system, the operator must manually configure the scanner before acquiring the Color Doppler data. Furthermore, the only direct way to get data out of the scanner is to capture the image displayed by the scanner and process the image pixel by pixel to determine velocity. It is presumed that with the support of a manufacturer a scanner could be adapted to integrate better with the envisioned detection and treatment system. Also, this scanner is not portable, although other truly portable, hand-carried ultrasound systems are available commercially.

The empirical system used an annular array HIFU transducer that only allows focusing in the axial direction. This is, however, the most important direction for focusing because the transducer must remain in contact with the skin. The detection algorithm in the empirical system operates on a line of pixels corresponding to the HIFU transducer's focal range. The algorithm could be modified to operate on all of the pixels in the ultrasound scanner's image, and after detecting the bleed instruct the operator to tilt or move the transducer while keeping it in contact with the skin and keeping the target within the transducer's focal range. In order to focus in other directions without manually moving the transducer, the transducer would either need to be mechanically-steered, or a linear array or two-dimensional array transducer would need to be used. Using a larger array, especially a two-dimensional array, would, however, increase the cost, complexity, and size of the device potentially making it less portable.

The detection algorithm presented here was implemented specifically to test the system with a string phantom and would not be appropriate for detecting true bleeds or blood flow in a vascular structure providing nutrients to an undesired tissue mass. Detecting and localizing a bleed with ultrasound, especially when combined with severe trauma, can challenge even the most skilled sonographer. Arterial bleeds have unique B-mode and Doppler signatures that can be exploited by a sonographer and also automated in software. Arterial bleeds are commonly associated with increased Doppler spectral broadening, increased systolic and diastolic flow, and decreased flow resistance. Blood flow through a puncture can result in turbulent flow that produces a characteristic speckled color pattern extending into the adjacent tissue when imaged with Color Doppler. Bleeding into a pseudo aneurysm is characterized by rapid forward and reverse flow in the neck and swirling flow in the cavity of the pseudo aneurysm.

In the empirical system, detection and targeting was performed autonomously, but the operator must start and stop HIFU delivery. Whether the ultimate system is partially or fully autonomous is likely to be determined by the level of expertise and setting in which the system is to be used. If used in the battlefield by soldiers other than field medics, a fully autonomous system might be preferred. In other settings (such as a clinical setting), a partially autonomous system might be preferred. In all settings, incorporation of autonomous treatment monitoring would be beneficial. Treatment could be initiated by an operator and automatically terminated when the bleed stops or other clinical objective is achieved with the option of ending treatment manually if the patient experiences excessive heat or pain during the procedure.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for using high intensity focused ultrasound (HIFU) to treat undesired tissue by damaging selected vascular regions to affect a viability of the undesired tissue, comprising the steps of:
(a) collecting ultrasound data from the undesired tissue that provides information about flow rates in vascular structures associated with the undesired tissue;
(b) presenting a combined display to a user, where the combined display includes:
(i) an ultrasound image of the undesired tissue and the associated vascular structures;
(ii) the flow rate information; and
(iii) a visualization of a focal point of a HIFU device;
(c) selecting a target site based on the flow rate data and the ultrasound image;
(d) automatically delivering HIFU therapy to the target site; wherein a duration of the therapy is controlled using a negative feedback loop provided by flow rate information at the target site collected during the HIFU therapy, such that the HIFU therapy is automatically terminated when the flow rate at the target site reaches a predetermined value; and
(e) frequently refreshing the combined display during the HIFU therapy, to enable the user to monitor the progress of the HIFU therapy in real-time.

2. The method of claim 1, wherein the predetermined value represents reducing the flow rate at the target site by at least about 90%.

3. The method of claim 1, wherein the predetermined value corresponds to a background signal associated with the undesired tissue.

4. The method of claim 1, wherein the step of selecting a target site is performed by the user, such that the user determines where the HIFU focal point should be positioned before HIFU therapy is initiated.

5. The method of claim 4, further comprising the step of enabling the user to determine a radius about the HIFU focal point corresponding to a volume to be automatically treated.

6. The method of claim 1, wherein the step of selecting a target site is performed by automatically, by a processor that analyzes the flow rate information to determine where the HIFU focal point should be positioned before HIFU therapy is initiated.

7. The method of claim 6, wherein if there are a plurality of vascular structures associated with the undesired tissue, the step of automatically selecting the target site comprises the step of:
(a) determining flow rate information for each such vascular structure; and
(b) identifying a vascular structure providing a largest flow rate into the undesired tissue as the target site.

8. The method of claim 6, wherein if there are a plurality of vascular structures associated with the undesired tissue, the step of automatically selecting the target site comprises the step of:
(a) determining flow rate information for each such vascular structure;
(b) ignoring each vascular structure corresponding to a flow rate below a predetermined value; and
(c) identifying each vascular structure providing a flow rate into the undesired tissue above a predetermined value as the target site, such that HIFU therapy is automatically performed at each target site so identified.

9. The method of claim 1, wherein the step of selecting a target site comprises the steps of:
(a) enabling the user to define a region of interest (ROI), where tissue within the ROI can be treated with HIFU without substantially damaging non-target tissue; and
(b) automatically selecting a target site within the region of interest, using a processor that analyzes the flow rate information to determine where the HIFU focal point should be positioned before HIFU therapy is initiated, that position corresponding to the target site.

10. The method of claim 1, wherein the step of presenting the combined display to the user further comprises displaying a relative HIFU dose during the HIFU therapy.

11. The method of claim 1, wherein the step of presenting the combined display to the user further comprises displaying a status of the HIFU beam, to enable the user to determine if the HIFU beam is energized.

12. A method for using high intensity focused ultrasound (HIFU) to treat undesired tissue by damaging selected vascular regions to affect a viability of the undesired tissue, comprising the steps of:
(a) collecting ultrasound data from the undesired tissue that provides information about flow rates in vascular structures associated with the undesired tissue;
(b) presenting a combined display to a user, where the combined display includes:
(i) an ultrasound image of the undesired tissue and the associated vascular structures;
(ii) the flow rate information; and
(iii) a visualization of a focal point of a HIFU device;
(c) selecting a target site based on the flow rate data and the ultrasound image; and
(d) automatically delivering HIFU therapy to the target site; wherein a duration of the therapy is controlled using a negative feedback loop provided by flow rate information at the target site collected during the HIFU therapy, such that the HIFU therapy is automatically terminated when the flow rate at the target site reaches a predetermined value.

13. A system for using high intensity focused ultrasound (HIFU) to treat undesired tissue by damaging selected vascular regions to affect a viability of the undesired tissue, comprising:
(a) an imaging ultrasound component for collecting ultrasound data from the undesired tissue to provide information about flow rates in vascular structures associated with the undesired tissue and an ultrasound image;
(b) a HIFU therapy component for delivering HIFU therapy to a target site;
(c) a user interface enabling a user to interact with the system;
(d) a display component for providing information to a user; and
(e) a controller implementing the following functions:
(i) generating a combined display on the display component, the combined display including an ultrasound image of the undesired tissue and the associated vascular structures, the flow rate information; and a visualization of a focal point of a HIFU device;
(ii) automatically selecting a target site based on the flow rate data and a user defined region of interest; and
(iii) automatically delivering HIFU therapy to the target site; wherein a duration of the therapy is controlled using a negative feedback loop provided by flow rate information at the target site collected during the HIFU therapy, such that the HIFU therapy is automatically terminated when the flow rate at the target site reaches a predetermined value.

14. The system of claim 13, wherein the controller automatically selects the target by:
(a) determining flow rate information for each vascular structure associated with the region of interest;
(b) ignoring each vascular structure corresponding to a flow rate below a predetermined value; and
(c) identifying each vascular structure providing a flow rate into the undesired tissue above a predetermined value as the target site, such that HIFU therapy is automatically performed at each target site so identified.

15. The system of claim 13, wherein the controller automatically selects the target by:
(a) determining flow rate information for each vascular structure associated with the region of interest; and
(b) identifying a vascular structure providing a largest flow rate into the undesired tissue as the target site.
